(19) United States
(12) Patent Application Publication
(10) Pub. No.: US 2017/0087200 A1
(43) Pub. Date: Mar. 30, 2017

(54) GREEN TEA EXTRACT COMPOSITION FOR HEPATIC FIBROSIS AND METHOD OF MAKING THE SAME

(71) Applicant: KUWAIT UNIVERSITY, SAFAT (KW)

(72) Inventor: Abdel-Majeed Safer, Bayan (KW)

(21) Appl. No.: 15/226,255

(22) Filed: Aug. 2, 2016

Related U.S. Application Data
(62) Division of application No. 14/872,070, filed on Sep. 30, 2015, now abandoned.

(51) Int. Cl. A61K 36/82; A61K 9/00; A61K 9/50

(52) U.S. Cl. CPC A61K 36/82; A61K 9/5036; A61K 9/0053

(57) ABSTRACT

The green tea extract composition for hepatic fibrosis includes green tea extract encapsulated in chitosan nanoparticles. In order to treat a patient suffering from hepatic fibrosis, the patient is administered a therapeutically effective dose of the green tea extract composition for hepatic fibrosis. The treatment is preferably administered orally. In order to make the green tea extract treatment, chitosan and green tea extract (prepared from Camellia sinensis) are mixed in de-ionized water to form a first solution, which is then stirred. Pentasodium triphosphate is added to the first solution to foam a second solution, which is then sonicated to form nanoparticles of green tea extract encapsulated in chitosan. Preferably, the second solution is further stirred for approximately two hours following the sonication.

### CROSS REFERENCE

This application is a divisional of U.S. application Ser. No. 14/872,070, filed Sep. 30, 2015, now pending.

### BACKGROUND OF THE INVENTION

1. Field of the Invention

The present invention relates to treatments for hepatic fibrosis, and particularly to a composition including green tea extract.

2. Description of the Related Art

Hepatic fibrosis is a liver disease that causes hepatic stellate cells to be over-active. This activity triggers extracellular matrix (ECM) synthesis and collagen fibers deposit in the extra-cellular spaces of the liver cells. During this process, blood infusion is lost and the tissue hardens, leading to fibrosis. Fibrosis itself causes no symptoms but can lead to portal hypertension or cirrhosis.

Due to the invasive nature of current therapies, there is a great deal of interest in developing relatively non-invasive therapies for reversing the fibrosis. At present, most conventional antifibrotic treatments are too toxic for long-teem use. Although silymarin, present in milk thistle, is a popular alternative medicine used to treat hepatic fibrosis, and appears to be safe, it appears to lack efficacy. It would be desirable to provide a safe and effective antifibrotic treatment for the prevention and treatment of hepatic fibrosis. Thus, a green tea extract composition for hepatic fibrosis addressing the aforementioned problems is desired.

### SUMMARY OF THE INVENTION

The green tea extract composition for hepatic fibrosis includes green tea extract encapsulated in chitosan nanoparticles. In order to treat a patient suffering from hepatic fibrosis, the patient is administered a therapeutically effective dose of the green tea extract composition for hepatic fibrosis. The treatment is preferably administered orally.

In order to make the green tea extract composition, chitosan and green tea extract (prepared from Camellia sinensis) are mixed in de-ionized water to form a first solution, which is then stirred. Pentasodium triphosphate is added to the first solution to faun a second solution, which is then sonicated to form nanoparticles of green tea extract encapsulated in chitosan. Preferably, the second solution is further stirred for approximately two hours following the sonication. The first solution is preferably approximately 47.5 wt % of de-ionized water, approximately 50.0 wt % of chitosan, and approximately 2.5 wt % of the green tea extract. The pentasodium triphosphate preferably forms approximately 4.76 wt % of the second solution.

These and other features of the present invention will become readily apparent upon further review of the following specification and drawings.

### BRIEF DESCRIPTION OF THE DRAWINGS

FIG. 1A is a scanning electron microscope image of rat liver extracellular matrix with collagen fiber formation due to exposure to carbon tetrachloride (CCl₄) and ethanol.

FIG. 1B is a scanning electron microscope image of rat liver extracellular matrix with collagen fiber formation due to exposure to carbon tetrachloride (CCl₄) and ethanol, similar to the sample of FIG. 1A, with additional treatment with the green tea extract composition for hepatic fibrosis according to the present invention.

FIG. 2A is a transmission electron microscope image of healthy hepatocytes of a first control group.

FIG. 2B is a transmission electron microscope image of a second group of hepatocytes exposed to carbon tetrachloride (CCl₄) and ethanol.

FIG. 2C is a transmission electron microscope image of a third group of hepatocytes exposed to carbon tetrachloride (CCl₄) and ethanol, and treated with chitosan.

FIG. 2D is a transmission electron microscope image of a fourth group of hepatocytes exposed to carbon tetrachloride (CCl₄) and ethanol, and treated with the green tea extract composition for hepatic fibrosis according to the present invention.

FIG. 3A is a scanning electron microscope image of green tea extract encapsulated in chitosan nanoparticles prepared according to the present method of making the green tea extract composition for hepatic fibrosis.

FIG. 3B is a graph showing a grey scale profile of a single green tea extract encapsulated in chitosan nanoparticle “A” of FIG. 3A.

FIG. 3C is a graph showing a grey scale profile of a single green tea extract encapsulated in chitosan nanoparticle “C” of FIG. 3A.

FIG. 3D is a graph showing a grey scale profile of a path through which the line profile of the cluster of green tea extract encapsulated in chitosan nanoparticles of FIG. 3A is computed.

Unless otherwise indicated, similar reference characters denote corresponding features consistently throughout the attached drawings.

### DETAILED DESCRIPTION OF THE PREFERRED EMBODIMENTS

The green tea extract composition for hepatic fibrosis includes green tea extract (GTE) encapsulated in chitosan nanoparticles. A method of preparing the green tea extract composition can include synthesizing chitosan nanoparticles using the ionic gelation technique with pentasodium triphosphate (TPP) as a crosslinking agent. For example, chitosan and GTE can be mixed together to form a first mixture and then pentasodium triphosphate (TPP) can be added to the first mixture to form a second mixture. The first mixture can include approximately 47.5 wt % de-ionized water, approximately 50.0 wt % chitosan, and approximately 2.5 wt % green tea extract. The second mixture can include approximately 4.76 wt % pentasodium triphosphate.

A method of treating a patient suffering from hepatic fibrosis can include administering to the patient a therapeutically effective amount of the green tea extract composition for hepatic fibrosis. A therapeutically effective amount of the green tea extract composition for hepatic fibrosis may be determined initially from in vivo assays described herein and adjusted for specific desired effectiveness using routine methods.

The green tea extract composition for hepatic fibrosis can be administered by any conventional route of administration, including, but not limited to, intravenous, oral, subcutaneous, intramuscular, intradermal and parenteral. Depending on the route of administration, the green tea extract composition for hepatic fibrosis can be constituted into any form. For example, forms suitable for oral administration include solid forms, such as pills, gelcaps, tablets, caplets, capsules (each including immediate release, timed release and sustained release formulations), granules, and powders. Forms suitable for oral administration also include liquid forms, such as solutions, syrups, elixirs, emulsions, and suspensions. In addition, forms useful for parenteral administration include sterile solutions, emulsions and suspensions.

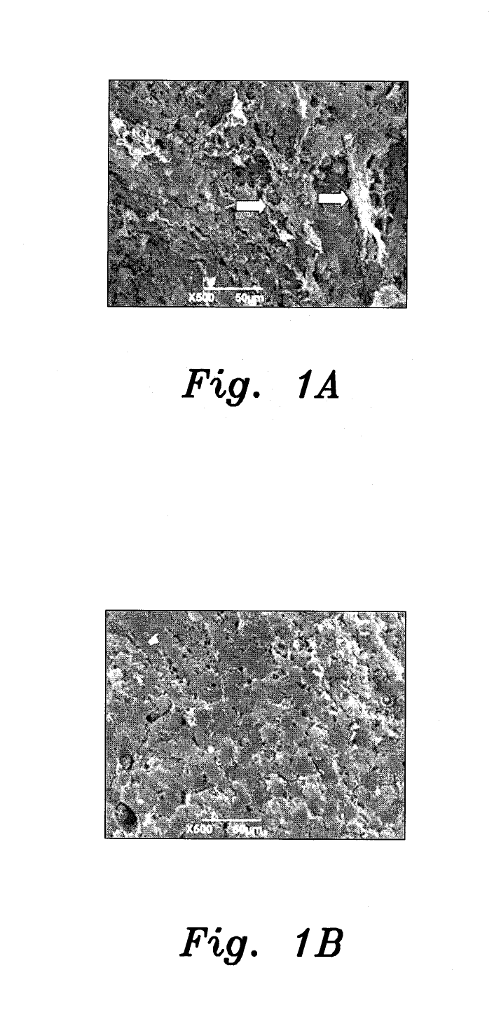

The present inventor has discovered that nanoparticles of green tea extract encapsulated in chitosan can effectively treat hepatic fibrosis. As will be discussed in greater detail below, rats were dosed with carbon tetrachloride (CCl₄) and ethanol in order to induce hepatic fibrosis. FIG. 1A is a scanning electron microscope image of rat liver extracellular matrix with collagen fiber formation due to exposure to carbon tetrachloride (CCl₄) and ethanol. The white arrows in FIG. 1A indicate formations of collagen fibers in the extracellular matrix. FIG. 1B is a scanning electron microscope image of rat liver extracellular matrix taken from the same sample group of rats dosed with carbon tetrachloride (CCl₄) and ethanol, however the rat's liver in FIG. 1B was also dosed with nanoparticles of green tea extract encapsulated in chitosan. FIG. 1B clearly shows a reduction in the presence of collagen fibers due to the therapeutic effect of the administered nanoparticles of green tea extract encapsulated in chitosan.

In order to prepare the green tea extract, 100 g of dried green tea leaves (Camellia sinensis) were powdered in a blender and extraction was performed with 1 L of double-distilled water at 85° C. for one hour. The extract was filtered through a nylon filter, and the filtrate was centrifuged at 3000 g for 15 minutes (where g is the gravitational acceleration at the Earth's surface; i.e., 9.8 m/s²). The clear supernatant was removed from the centrifuge and the residual pellet was shaken with distilled water (warmed to 35° C.) and centrifuged again. The supernatants were then pooled, lyophilized, and the resulting material was stored at −20° C. in a screw-capped bottle.

The green tea extract encapsulated in chitosan nanoparticles (GTE-CS NPs) were prepared by synthesizing chitosan nanoparticles using the ionic gelation technique with pentasodium triphosphate (TPP) as a crosslinking agent. 9.5 mL of de-ionized water, 500 μL of chitosan (20 mg/mL) and 100 μL of GTE (5 mg/mL) were added together and stirred for about an hour. 100 μL of TPP (10 mg/mL) was then added, drop by drop, with constant stirring. The entire solution was then sonicated for about 30 seconds using a probe sonicator and stirred for another two hours.

As noted above, liver fibrosis was induced in a group of rats with dual exposure to carbon tetrachloride (CCl₄) and ethanol. Administration of alcohol, with repetitive CCl₄ ingestion, enhanced the toxicity of CCl₄ in the rat model. Other groups of rats were treated with CCl₄ alone, ethanol alone, and one group was left untreated. Specifically, four groups of male Sprague Dawley rats, each weighing 200-250 g, were used in the study. In each group, the delivery vehicle was physiological saline. Group 1, the control group, received the vehicle alone. Group 2 received subcutaneous injections of 40% CCl₄ and/or ethanol in vehicle solution for three weeks. Group 3 received subcutaneous injections of 40% CCl₄ and/or ethanol in vehicle solution for three weeks, and then were treated orally with the present nanoparticles of green tea extract encapsulated in chitosan for 25 days. Group 4 was dosed with ethanol alone, without CCl₄, and received nanoparticles of green tea extract encapsulated in chitosan for 25 days, delivered orally.

After 25 days, during autopsy, the rat livers which had been dosed with CCl₄ showed a typical fibrotic orange color, as opposed to a normal reddish brown color. The weights of the animals steeply declined with the onset of CCl₄ treatments, as expected, and gradually went up in Group 3, as administration of the nanoparticles of green tea extract encapsulated in chitosan caused the rats to gain back lost weight. By the end of the experiment, at week four, almost all rats from Group 3 were back to normal body weights, close to those of Group 1, the control group (with p<0.05). The weights of the rats dosed solely with ethanol were found to increase, and a gradual decrease was observed in Group 3, following administration of the nanoparticles of green tea extract encapsulated in chitosan, restoring their weights to be in-line with the control group (with p<0.05).

For the CCl₄ sub-group of Group 3, treated with the nanoparticles of green tea extract encapsulated in chitosan, the rats showed normal growth (r=0.5, p=0.009). In the sub-group treated with ethanol alone (Group 2), the weight increased rapidly over the experimental period (r=0.41, p=0.01), and in the sub-group of Group 3 receiving only ethanol, but treated with the nanoparticles of green tea extract encapsulated in chitosan, the rat weights increased with over the time of the experiment (r=0.18, p=0.27). Rats treated with both ethanol and CCl₄ showed a decrease in weight with time, as a result of the CCl₄ effect, but this effect was slightly reversed by the ethanol, resulting in a non-significant decrease in weight (r=−0.20, p=0.25). The effect of the nanoparticles of green tea extract encapsulated in chitosan on the ethanol+CCl₄ group showed a shift in weight back to normal weight (r=−0.02, p=0.92).

After a period of 25 days, just prior to dissection, the rats treated with CCl₄ alone, ethanol alone, and particularly the combination of CCl₄ and ethanol, appeared fragile and thin with pale yellow hair. Both the control group of rats and those treated with the present green tea extract composition appeared healthy. Histological observation of liver tissues in hematoxylin and eosin stain (H&E stain) and toluidine blue stained sections all coincided with the external status of the animals and the autopsy features. Histopathological changes were clear in the H&E and toluidine blue liver sections, such as destruction of lobular architecture, inflammation, large foamy vacuolated cytoplasm, necrosis, large fatty cells, steatosis, nuclear shrinkage, abnormal tri-polar and tetra-polar divisions, nuclear karyorrhesis, nuclear karyolysis, nuclear hyperchromatism, dead cells, thickening of portal vein and portal triad, hypertension of arterioles, nuclear hyperchromatism, nuclear fragmentation, condensed eiosenophilic protein, hyperactive Kuppfer cells, and proliferation of hepatic stellate cells (HSCs).

Most of the above pathological features were noticeably reduced after administration of the nanoparticles of green tea extract encapsulated in chitosan. Primarily, cytoplasmic vacuolation and the large fatty cells disappeared in the samples treated with the nanoparticles of green tea extract encapsulated in chitosan (not only those seen in gross morphology, but also those observed in the H&E and toluidine blue stained sections). Masson's trichrome stained liver tissues clearly showed the intermingled fibrous materials in the CCl₄+ethanol-treated liver (shown as blue-green fibrous structures among the cells) and around the blood vessels. Such fibers were not present in the samples which had been treated with the nanoparticles of green tea extract encapsulated in chitosan, and visually appeared like those of the control group. Surface topography of the fractured surface of the liver blocks, when observed under scanning electron microscope (SEM), also showed how various types of fibers, both in thickness and direction, intermingled in the liver parenchyma around the hepatocytes.

During hepatic fibrosis, besides other proteins, collagen types I and III mostly proliferate. Administration of the nanoparticles of green tea extract encapsulated in chitosan, either simultaneously with or following CCl₄/ethanol/CCl₄+ethanol treatment, was found to prevent hepatic fibrosis. This indicates that the nanoparticles of green tea extract encapsulated in chitosan inhibit proliferation of hepatic stellate cells. All groups treated with the nanoparticles of green tea extract encapsulated in chitosan showed a significant effect of destroying almost all fibers seen in an area comparable to that of the CCl₄+ethanol group.

Importantly, two kinds of fibrous materials were seen: 312.41 nm thick (on average) fibers were always visible in CCl₄-treated liver samples, and 169.71 nm thin, fluffy fibers (on average) always appeared in ethanol-treated liver samples, in comparison to the normal range of 800 nm to 2400 nm, indicating hepatic injury, and these counterparts were noticeably reduced when treated with the nanoparticles of green tea extract encapsulated in chitosan.

The nanoparticles of green tea extract encapsulated in chitosan inhibit the damaging effects caused by the oxidative stress of CCl₄, ethanol, and the combination of CCl₄ and ethanol on the liver cells. Thus, the nanoparticles of green tea extract encapsulated in chitosan significantly reduced cellular leakage of hepatocyte aminotransferases (AST and ALT), and further apparently improved cell viability. Further, severe hepatic lesions, induced by the dual action of CCl₄ and ethanol, were markedly improved by the administration of the nanoparticles of green tea extract encapsulated in chitosan. Additionally, the nanoparticles of green tea extract encapsulated in chitosan reduced inflammation and destruction of liver architecture and down-regulation of PDGF-beta receptor, thus preventing the development of CCl₄+ethanol-induced hepatic fibrosis in rats. This suggests that the green tea's polyhydroxy phenolics (catechins) exhibit strong antioxidant activity against reactive oxygen species (ROS), and have beneficial health effects in curing both structure and function of hepatic extracellular matrix (ECM) to a significant degree. This is primarily through epigallocatechin gallate (EGCG), the active ingredient in green tea that inhibits the activation of receptor tyrosine kinases (RTKs) associated with HSCs.

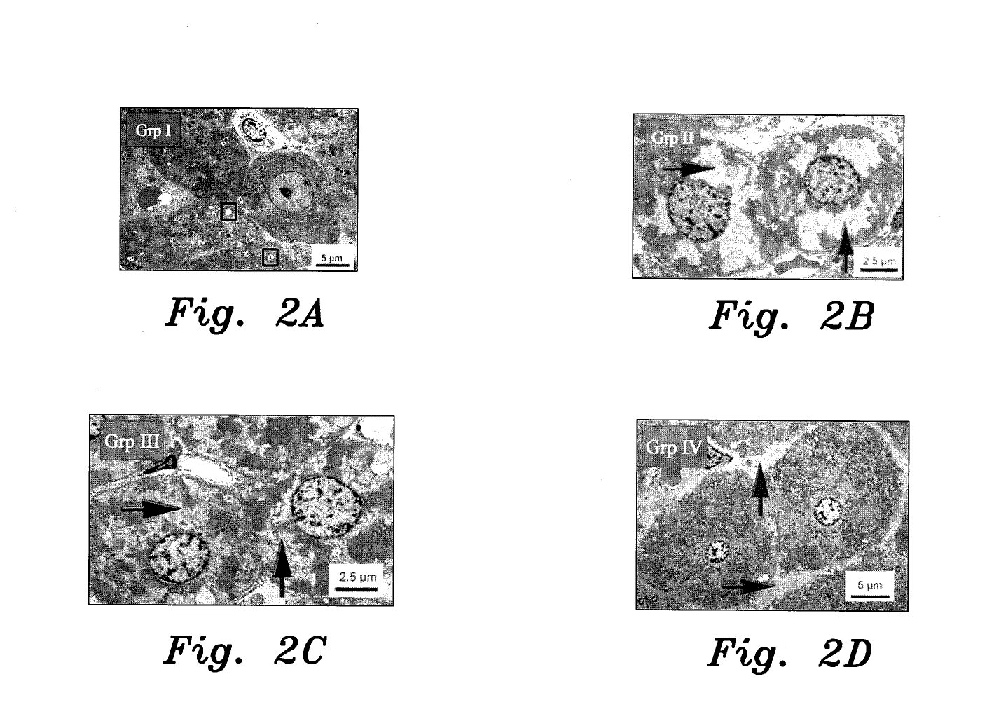

FIG. 2A is a transmission electron microscope (TEM) image of healthy hepatocytes taken from Group 1; i.e., the control group, as discussed above. FIG. 2B is a TEM image of hepatocytes from Group 2, which were exposed to carbon tetrachloride (CCl₄) and ethanol. In FIG. 2B, the damaged cytoplasm, caused by the treatment with CCl₄ and ethanol, can be clearly seen (indicated by the black arrows in FIG. 2B). FIG. 2C is a TEM image of a third group of hepatocytes exposed to CCl₄ and ethanol, and treated only with chitosan. As indicated by the black arrows in FIG. 2C, the damaged cytoplasm still exists (similar to that shown in FIG. 2B), indicating that chitosan alone does not aid in the healing process. FIG. 2D is a TEM image of Group 3 hepatocytes, which were exposed to CCl₄ and ethanol, and then treated with the present green tea extract treatment for hepatic fibrosis. As indicated by the black arrows in FIG. 2D, treatment with the nanoparticles of green tea extract encapsulated in chitosan clearly restores the hepatocytes to their normal morphological structures.

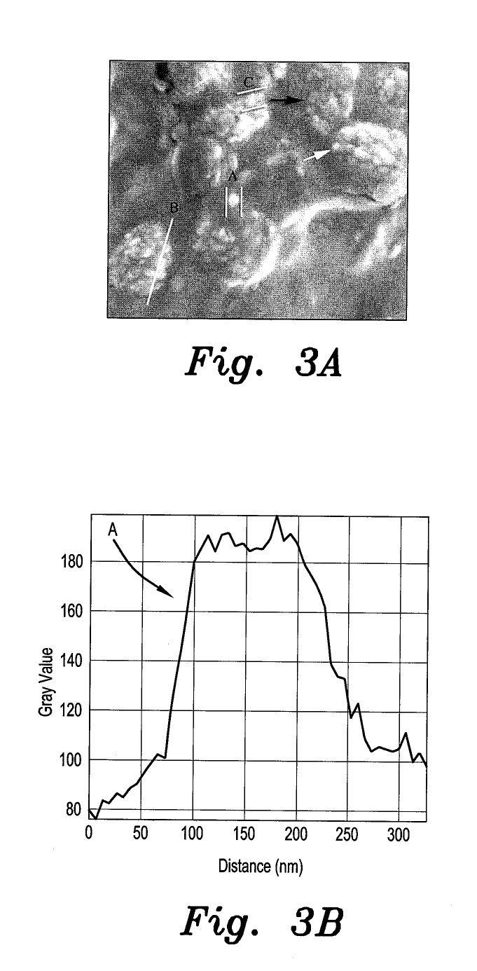

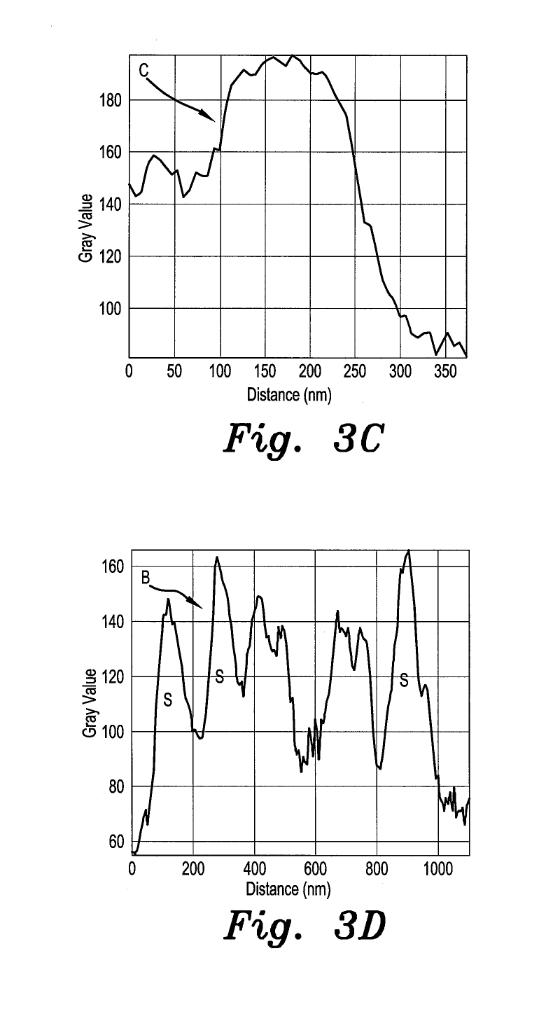

The size of the nanoparticles of green tea extract encapsulated in chitosan was determined by both zeta potential measurement and scanning electron microscopy. FIG. 3A is an SEM image of green tea extract encapsulated in chitosan nanoparticles prepared according to the above method. FIG. 3B is a graph showing the grey scale profile of the single green tea extract encapsulated in chitosan nanoparticle “A” of FIG. 3A. FIG. 3C is a graph showing the grey scale profile of the single green tea extract encapsulated in chitosan nanoparticle “C” of FIG. 3A. From FIGS. 3A, 3B and 3C, one can see that each nanoparticle of green tea extract encapsulated in chitosan has a diameter ranging from approximately 100 nm to approximately 200 nm, with the peaks in FIGS. 3B and 3C occurring at approximately 175 nm FIG. 3D is a graph showing the grey scale profile of path “B”, through which the line profile of the cluster of green tea extract encapsulated in chitosan nanoparticles of FIG. 3A is computed. In FIG. 3D, the regions “S” represent profiles of smaller nanoparticles which are either broken or buried in the specimen.

It is to be understood that the present invention is not limited to the embodiments described above, but encompasses any and all embodiments within the scope of the following claims.

1-3. (canceled)

4. A method of treating hepatic fibrosis, comprising the step of administering to a patient suffering from hepatic fibrosis a therapeutically effective amount of a green tea extract composition for hepatic fibrosis, the green tree extract composition for hepatic fibrosis including nanoparticles of green tea extract encapsulated in chitosan, wherein the diameter of the encapsulated nanoparticles of the green tea extract is about 175 nm.

5. The method of treating hepatic fibrosis as recited in claim 4, wherein the green tea extract composition for hepatic fibrosis is administered to the patient orally.

6-17. (canceled)
